(19) United States
(12) Patent Application Publication
(10) Pub. No.: US 2025/0186787 A1
(43) Pub. Date: Jun. 12, 2025

(54) Sub-Perception Spinal Cord Stimulation at Low Frequencies

(71) Applicant: Boston Scientific Neuromodulation Corporation, Valencia, CA (US)

(72) Inventors: Kellan Onuscheck, Dallas, TX (US); Luke Barnes, Northlake, TX (US)

(21) Appl. No.: 18/949,609

(22) Filed: Nov. 15, 2024

Related U.S. Application Data
(60) Provisional application No. 63/606,901, filed on Dec. 6, 2023.

(51) Int. Cl. A61N 1/372; A61N 1/36

(52) U.S. Cl. CPC A61N 1/37247; A61N 1/36062; A61N 1/36171; A61N 1/36175; A61N 1/36185

(57) ABSTRACT

Algorithms for determining optimal low-frequency and sub-perception spinal cord stimulation for a patient are disclosed. A spread bipole is preferably used both to position the stimulation to target a patient's pain, and to provide the resulting therapy. The stimulation is provided at very low frequencies, such as 10 Hz or less, and preferably 2 Hz. Further, sub-perception is provided by the use of lower amplitudes and at longer pulse widths than are conventional in typical SCS stimulation therapies. This results in determined stimulation therapies which draw very little power. Therapy is preferably provided by the use of symmetric biphasic pulses.

### CROSS REFERENCE TO RELATED APPLICATIONS

This is a non-provisional of U.S. Provisional Patent Application Ser. No. 63/606,901, filed Dec. 6, 2023, which is incorporated herein by reference, and to which priority is claimed.

### FIELD OF THE INVENTION

This application relates to Implantable Spinal Cord Stimulator (SCS) devices.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system. However, the present invention may find applicability with any stimulator device system.

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads 15 that form an electrode array 17. For example, one or more percutaneous leads 15 can be used having ring-shaped or split-ring electrodes 16 carried on a flexible body 18. In another example not illustrated, a paddle lead can provide an electrode array 17 positioned on one of its generally flat surfaces. Lead wires 20 within the leads are coupled to the electrodes 16 and to proximal contacts 22 insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins to stimulation circuitry 28 (FIG. 3) within the case 12.

In the illustrated IPG 10, there are sixteen electrodes (E1-E16), split between two percutaneous leads 15, and thus the header 23 may include two lead connectors 24. However, the type and number of leads, and the number of electrodes, and the number of lead connectors in an IPG is application specific and therefore can vary. The conductive case 12, or some conductive portion of the case, can also comprise an electrode (Ec). In an SCS application, the electrode lead(s) 15 are typically implanted in the spinal column inside the patient's vertebrae and proximate to the dura in a patient's spinal cord, preferably spanning left and right of the patient's spinal column. The proximal contacts 22 are tunneled through the patient's tissue to a distant location such as the buttocks where the IPG case 12 is implanted, at which point they are coupled to the lead connectors 24. SCS therapy is traditionally used to relieve symptoms such as chronic back pain. IPG 10 as described should be understood as including non-implantable External Trial Stimulators (ETSs), which mimic operation of the IPG 10 to provide trial stimulation during an external trial period when leads have been implanted in the patient but the IPG 10 has not yet been implanted. An ETS is connected to the implanted leads percutaneously. Sec, e.g., U.S. Pat. No. 9,259,574 (disclosing an ETS).

IPG 10 can include an antenna 26a allowing it to communicate bi-directionally with a number of external devices discussed subsequently. Antenna 26a as shown comprises a conductive coil within the case 12, although this coil antenna can also appear in the header 23. When antenna 26a is configured as a coil, communication with external devices preferably occurs using near-field magnetic induction. IPG 10 may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30), as shown in the example of FIG. 2. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2, electrode E1 has been selected as an anode (during its first phase 30a), and thus provides pulses which source a positive current of amplitude +I to the tissue. Electrode E2 has been concurrently selected as a cathode (again during first phase 30a), and thus provides pulses which sink a corresponding negative current of amplitude −I from the tissue. This is an example of bipolar stimulation, in which the electrode array 17 includes one anode pole and one cathode pole. Stimulation provided by the IPG 10 can also be monopolar, with the electrode array 17 programmed with a single pole of a given polarity (e.g., a cathode pole), and with the conductive case electrode Ec acting as a return (e.g., an anode pole). Multipolar (e.g., tripolar) stimulation can also be used, with the electrode array 17 having three or more poles. Note that more than one electrode in the electrode array 17 may be active to form a pole, as discussed and shown further below. See also U.S. Pat. No. 10,881,859, which is incorporated herein by reference in its entirety.

IPG 10 as mentioned includes stimulation circuitry 28 to form prescribed stimulation at a patient's tissue, and FIG. 3 shows an example of such circuitry. The stimulation circuitry 28 shown includes one or more current source circuits and one or more current sink circuits. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is associated with an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient's tissue, R, hopefully with good therapeutic effect. Consistent with the example provided in FIG. 2, FIG. 3 shows operation during the first phase 30a in which electrode E1 has been selected as an anode electrode to source current I to the tissue R and E2 has been selected as a cathode electrode to sink current I from the tissue. Thus PDAC1 and NDAC2 are digitally programmed (Ip1, In2) to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16. Other stimulation circuitries 28 can also be used in the IPG 10, including ones that include switching matrices between the electrode nodes ci 39 and the N/PDACs. See, e.g., U.S. Pat. Nos. 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ci 39 can be integrated on one or more Application Specific Integrated Circuits (ASICs), as described in U.S. Patent Application Publications 2012/0095529, 2012/0092031, and 2012/0095519. As explained in these references, ASIC(s) may also contain, or be coupled with, other circuitry useful in the IPG 10, such as a microcontroller, telemetry circuitry (for interfacing off chip with telemetry antennas 26a and/or 26b), circuitry for generating the compliance voltage VH which powers the stimulation circuitry 28, various measurement circuits, etc. Collectively, such circuitry comprises control circuitry in the IPG 10.

Also shown in FIG. 3 are DC-blocking capacitors Ci 38 placed in series in the electrode current paths between each of the electrode nodes ci 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861. While useful, DC-blocking capacitors 38 are not strictly required in all IPG designs and applications.

Referring again to FIG. 2, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. During the first pulse phase 30a (e.g., PDAC1 and NDAC2 are activated), charge will (primarily) build up across the DC-blockings capacitors (e.g., C1 and C2) associated with the electrodes (e.g., E1 and E2) used to produce the current. During the second pulse phase 30b, when the polarity of the current I is reversed at the selected electrodes E1 and E2 (e.g., PDAC2 and NDAC1 are activated), the stored charge on capacitors C1 and C2 is recovered.

In the example waveform in FIG. 2, the biphasic pulses are symmetric, meaning that the second phase 30b is of equal size and shape as the first phase 30a, but of opposite polarity. That, is, the amplitude and pulse widths of these phases 30a and 30b have equal magnitudes. A biphasic pulses does not need to be symmetric to provide charge recovery; ideal charge recovery will occur if the areas of the two phases 30a and 30b are the same (e.g., |I|*PW, even if I and PW are different in these phases).

Charge recovery using phases 30a and 30b is said to be “active” because the P/NDACs in stimulation circuitry 28 actively drive a current, in particular during second phase 30b to recover charge stored after the first phase 30a. However, such active charge recovery may not be perfect, and some residual charge may be present in capacitive elements in the current path even after phase 30b is completed. Accordingly, the stimulation circuitry 28 can also provide for passive charge recovery. Passive charge recovery is implemented using passive charge recovery switches 41 as shown in FIG. 3. These switches 41 when selected via assertion of control signals Xi couple each electrode node ei to a particular circuit node (shown here as the battery voltage Vbat, although another DC node could be used as well). As explained in U.S. Pat. Nos. 10,716,937 and 10,792,491, this allows any stored charge to be passively recovered through the patient's tissue, R. Control signals Xi are usually asserted to cause passive charge recovery after each pulse (e.g., after phase 30b) during periods 30c shown in FIG. 2, and are at least asserted in the previously active current paths: that is, at least X1 and X2 would be asserted in the example of FIG. 2 (although all control signals Xi could also be asserted). Because passive charge recovery involves capacitive discharge through the resistance R of the patient's tissue, such discharge manifests as an exponential decay in current, although this is not shown in FIG. 2. Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

Although not shown in FIG. 2, stimulation pulses can also be monophasic, having only a single actively driven phase (30a). Because monophasic pulses lack an active charge recovery phase (30b), such monophasic pulses would typically be followed by passive charge recovery (30c) as just described.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 26a in the IPG 10. The external controller 60 can also have a far-field RF antenna 64b capable of wirelessly communicating with the RF antenna 26b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 26a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 26b, the wand 76, the computing device, or both, can likewise include an RF antenna 74b to establish communication with the IPG 10 at larger distances. The clinician programmer 70 can also communicate with other devices and networks, such as the Internet, cither wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87. The external system software 96 can comprise instructions stored in a non-transitory computer readable medium, such as in solid state, magnetic, or optical memory stored in or with the external system, or a portable memory (e.g., a memory stick or disk), or in a server such as 86.

### SUMMARY

A method is disclosed for programming a spinal cord stimulator using an external system in communication with the spinal cord stimulator, the spinal cord stimulator comprising an electrode array comprising a plurality of electrodes implanted in a spinal column of a patient. The method may comprise: (a) accessing a graphical user interface of the external system to cause the spinal cord stimulator to provide stimulation pulses of a first amplitude and a first pulse width from at least one electrode in the electrode array, wherein the stimulation pulses are sub-perception for the patient; (b) accessing the graphical user interface to cause the spinal cord stimulator to increase the first pulse width to a second pulse width where the stimulation pulses are supra-perception for the patient; (c) accessing the graphical user interface to cause the spinal cord stimulator to decrease the first amplitude to a second amplitude where the stimulation pulses are sub-perception for the patient; and (d) providing therapeutic sub-perception stimulation to the patient via the stimulation pulses at the second amplitude and at the second pulse width.

In one example, the stimulation pulses have a first frequency of 10 Hz or less. In one example, the first frequency is 2 Hz or less. In one example, the stimulation pulses form a bipole in the electrode array. In one example, the method further comprises, before step (a), accessing the graphical user interface of the external system to cause the spinal cord stimulator to provide stimulation pulses which are supra-perception for the patient. In one example, the method further comprises moving the bipole in the electrode array. In one example, the bipole is moved longitudinally in the electrode array to address a symptom of the patient. In one example, the bipole is moved laterally in the electrode array to a point where the patient perceives symmetric stimulation on a left and right side of his body. In one example, the stimulation pulses form a spread bipole configured to approximate a linear electric field in tissue of the patient. In one example, the stimulation pulses comprise biphasic pulses comprising a first phase of a first polarity and a second phase of a second polarity. In one example, the first and second phases are symmetric. In one example, the first and second phases are actively driven by stimulation circuitry in the spinal cord stimulator. In one example, the steps are performed in order. In one example, in step (b), if the second pulse width is maximized before the stimulation pulses are supra-perception for the patient, adjusting the first amplitude to a higher value where the stimulation pulses are supra-perception for the patient. In one example, in step (b), if the second pulse width is maximized before the stimulation pulses are supra-perception for the patient, adjusting the first amplitude to a higher value, setting the pulse width to the first pulse width, and repeating step (b). In one example, performance of the method is enabled via selection of an option of the graphical user interface. In one example, in step (c) the first amplitude is decreased to the second amplitude by a pre-determined amount. In one example, the second amplitude comprises a fraction of the first amplitude. In one example, step (d) comprises providing therapeutic sub-perception stimulation to the patient via the stimulation pulses at the second amplitude, the second pulse width, and the first frequency. In one example, the method further comprises programming a patient external controller with the therapeutic sub-perception stimulation. In one example, the patient external controller is configured to allow the patient to adjust the second amplitude of the therapeutic sub-perception stimulation, wherein adjustment of the second amplitude is limited to a maximum comprising the first amplitude. In one example, the method further comprises analyzing at the external system a power indicative of the therapeutic sub-perception stimulation.

An external system configured for communication with a spinal cord stimulator is disclosed, the spinal cord stimulator comprising an electrode array comprising a plurality of electrodes implanted in a spinal column of a patient, the external system comprising a graphical user interface and control circuitry configured to: (a) receive via the graphical user interface a first input to cause the spinal cord stimulator to provide stimulation pulses of a first amplitude and a first pulse width from at least one electrode in the electrode array, wherein the stimulation pulses are sub-perception for the patient; (b) receive via the graphical user interface a second input to cause the spinal cord stimulator to increase the first pulse width to a second pulse width where the stimulation pulses are supra-perception for the patient; and (c) receive via the graphical user interface a third input to cause the spinal cord stimulator to decrease the first amplitude to a second amplitude where the stimulation pulses are sub-perception for the patient, whereby the spinal cord stimulator provides therapeutic sub-perception stimulation for the patient via the stimulation pulses at the second amplitude and at the second pulse width.

In one example, the stimulation pulses have a first frequency of 10 Hz or less. In one example, the first frequency is 2 Hz or less. In one example, the stimulation pulses are configured to form a bipole in the electrode array. In one example, the control circuitry is further configured, before step (a), to receive via the graphical user interface a fourth input to cause the spinal cord stimulator to provide stimulation pulses which are supra-perception for the patient. In one example, the control circuitry is further configured to receive via the graphical user interface a fifth input to move the bipole in the electrode array. In one example, the fifth input is configured to move the bipole longitudinally in the electrode array to address a symptom of the patient. In one example, the fifth input is configured to move the bipole laterally in the electrode array to a point where the patient perceives symmetric stimulation on a left and right side of his body. In one example, the stimulation pulses are configured to form a spread bipole configured to approximate a linear electric field in tissue of the patient. In one example, the stimulation pulses are configured as biphasic pulses comprising a first phase of a first polarity and a second phase of a second polarity. In one example, the first and second phases are symmetric. In one example, the first and second phases are configured to be actively driven by stimulation circuitry in the spinal cord stimulator. In one example, in step (b), if the second pulse width is maximized before the stimulation pulses are supra-perception for the patient, the control circuitry is further configured to receive via the graphical user interface a sixth input to adjust the first amplitude to a higher value where the stimulation pulses are supra-perception for the patient. In one example, in step (b), if the second pulse width is maximized before the stimulation pulses are supra-perception for the patient, the control circuitry is further configured to receive via the graphical user interface a seventh input to adjust the first amplitude to a higher value, and to set the pulse width to the first pulse width, before returning to step (b). In one example, in step (c) the third input is configured to decrease the first amplitude the second amplitude by a pre-determined amount. In one example, the second amplitude comprises a fraction of the first amplitude. In one example, in step (d) the spinal cord stimulator provides therapeutic sub-perception stimulation for the patient via the stimulation pulses at the second amplitude, the second pulse width, and at the first frequency. In one example, the external system further comprises a patient external controller, wherein the control circuitry is further configured to program the patient external controller with the therapeutic sub-perception stimulation. In one example, the control circuitry is further configured to limit a patient's adjustment of the second amplitude using the patient external controller to a maximum comprising the first amplitude. In one example, the control circuitry is further configured to analyze a power indicative of the therapeutic sub-perception stimulation.

A spinal cord stimulator device programmed to provide the therapy as disclosed herein and as determined with the disclosed algorithms is also within the scope of the invention.

### BRIEF DESCRIPTION OF THE DRAWINGS

FIG. 1 shows an Implantable Pulse Generator (IPG), in accordance with the prior art.

FIG. 2 shows an example of stimulation pulses producible by the IPG, in accordance with the prior art.

FIG. 3 shows stimulation circuitry useable in the IPG, in accordance with the prior art.

FIG. 4 shows various external devices capable of communicating with and programming stimulation in an IPG, in accordance with the prior art.

FIG. 5A shows a GUI present on an external system to program simulation in an IPG, with FIG. 5B showing an example of waveforms specified by the GUI, in accordance with the prior art.

FIGS. 6 and 7 shows different examples of an algorithm used to determine optimal sub-perception stimulation for an SCS patient.

### DETAILED DESCRIPTION

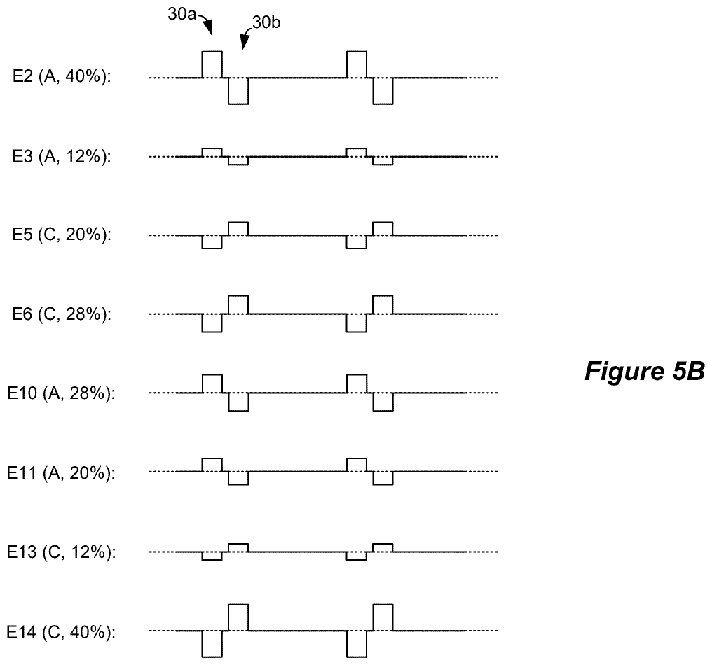

FIG. 5A shows a GUI 99 as may be rendered on an external system (60, 70, or 80) to program the stimulation the IPG 10 provides. The GUI 99 includes a waveform interface 100 which allows certain stimulation parameters (pulse amplitude I, pulse width PW, and frequency F) to be set or adjusted. Waveform interface 100 can include other options that define the basic shape of the stimulation waveform, like whether bipolar or monopolar pulses are used, whether biphasic or monophasic pulses are used, whether passive recovery is to be used, etc.

GUI 99 also includes an electrode configuration interface 102 which allows for the selection of electrodes 16 in the electrode array 17 that will provide the stimulation. Interface 102 as shown allows a user to select whether an electrode will operate as an anode, a cathode, or be off (inactive). Further, the percentage of the amplitude (X %) at each active electrode can be specified. In the example shown, electrodes E2, E3, E10, and E11 have been selected to act as anodes, with these electrodes receiving 40, 12, 28, and 20% of the amplitude I respectively as an anodic current. That is, E2 will provide 0.4*+I, E3 will provide 0.12*+I, etc. Electrodes E5, E6, E13, and E14 have been selected to act as cathodes, with these electrodes receiving 20, 28, 12, and 40% of the amplitude I respectively as a cathodic current. That is, E5 will provide 0.2*−I, E6 will provide 0.28*−I, etc. Examples of these waveforms at each of the active electrodes and their relative amplitudes are shown in FIG. 5B, and are shown using symmetric biphasic pulses with a first phase 30a having the polarity specified in interface 102 and a second phase 30b of opposite polarity.

GUI 99 in this example also includes a visualization interface 104. Preferably, this interface 104 shows the positioning of leads 15 in the electrode array 17 relative to each other as they are implanted in the patient, and relative to certain tissue structures in the patient such as various vertebrae (thoracic vertebrae T8-T12 are shown). Other relevant tissue structures could be shown in interface 104 as well. In the interface 104, the positioning of the leads 15 relative to tissue structures, as well as the position of the leads 15 relative to each other, preferably comes from imaging information (e.g., fluoroscopy) taken from the patient.

The visualization interface 104 can also preferably show some indication of the stimulation being provided. For example, different shading can be used to show which electrodes have been selected to act as anodes (dark), cathodes (grey), or that are off (light). Furthermore, a position of the poles formed by the active electrodes can also be shown. For example, because electrodes E2, E3, E10, and E11 act as anodes, they establish an anode pole (A, +) at a position in the electrode array 17 influenced by the magnitudes of the anodic current provided at these electrodes (i.e., in between them). Similarly, because electrodes E5, E6, E13, and E14 act as cathodes, they establish a cathode pole (C, −) at a position influenced by the magnitudes of the cathodic current provided at these electrodes. These magnitudes (e.g., X %) can also be displayed in the visualization interface 104, although this isn't shown.

As explained earlier and as shown in FIG. 5A, a pole can be formed in the electrode array 17 using one or more active electrodes (here, four electrodes are used to make each of the anode pole and the cathode pole). This can establish the poles as virtual poles at X-Y positions which may not correspond to the position of the physical electrodes 16 in the electrode array 17. The example of FIG. 5A also illustrates bipolar stimulation, which involves use of a single anode (+) and cathode (−) pole in the electrode array 17. The general position of the stimulation provided by the pole(s) in the electrode array can also be quantified. In the illustrated example, this position is midway between the anode pole and the cathode pole, and is called the center point of stimulation (CPS).

As discussed further in U.S. Pat. No. 10,881,859, an electrode configuration algorithm operable in or with the external system rendering GUI 99 can be used to determine the position of the poles and/or the CPS in the electrode array 17 given the selection of the electrodes in the electrode configuration interface 102. This algorithm can also operate in reverse. For example, a user can position the stimulation (the anode and/or cathode poles) in the electrode array 17 in the visualization interface 104 (using arrows 106, a mouse cursor for example), with the electrode configuration algorithm then operating in reverse to determine in electrode configuration interface 102 which electrodes should be active, and with which polarities and amplitude percentages, to form the poles at the specified positions.

Also shown in GUI 99 is an option 108 to form the stimulation as a spread bipole with a given focus (L). Examples of spread bipoles, and how they may be formed and configured, are disclosed in U.S. Pat. Nos. 10,549,097 and 11,376,433, and U.S. Patent Application Publication 2022/0296902, which are incorporated herein by reference in their entireties. A spread bipole is one which is relatively large in size, and the previously described bipole (as illustrated in interface 104) provides an example. In a spread bipole, current fractionalizations are provided at the electrodes in manner that approximates a linear electric field over the extent of the size of the bipole. This allows a spread bipole to provide a constant stimulation to a relatively large area or volume of tissue that is affected by that bipole.

This is beneficial in the disclosed spinal cord stimulation techniques, because a larger area or volume will more easily target a source of pain in the patient's tissue requiring stimulation is more easily recruited when a larger spread bipole is used. Thus, a spread bipole is easier to position in the electrode array 17 in a manner that will cover a source of pain in the patient's tissue than when a smaller bipole is used. Use of a spread bipole is also more likely to continue to target a patient's pain in the future. This is significant, because the leads 15 in the electrode array can migrate in position over time in the patient's tissue, either because of settling of the leads 15 in the spinal column, or because of patient movement. A larger spread bipole will likely continue to target the source of pain in the patient's tissue even if the leads migrate somewhat, meaning that such migration will likely not result in a loss of therapy. When a smaller bipole is used to provide therapy by contrast, lead migration may move the therapy away from the source of pain, meaning effective therapy will be lost.

The electrode configuration algorithm described earlier can operate when spread bipole option 108 is selected to define the necessary anodic and cathodic currents at the electrodes to form the spread bipole with the specified focus L. Focus distance L generally defines the length between the anode and cathode poles of the spread bipole, and can also be entered by the user at option 108. Once the spread bipole is formed in the electrode array 17, it can be moved (e.g., arrows 106) in X-Y directions in the electrode array 17, which the electrode configuration algorithm operating automatically to update the active electrodes, their polarities, and their relative percentages, in a manner to affect the desired movement.

While Spinal Cord Stimulation (SCS) therapy can be an effective means of alleviating a patient's pain, such stimulation can also cause paresthesia. Paresthesia—sometimes referred to a “supra-perception” therapy—is a sensation such as tingling, prickling, heat, cold, etc. that can accompany SCS therapy. Generally, the effects of paresthesia are mild, or at least are not overly concerning to a patient. Moreover, paresthesia is generally a reasonable tradeoff for a patient whose chronic pain has now been brought under control by SCS therapy. Some patients even find paresthesia comfortable and soothing.

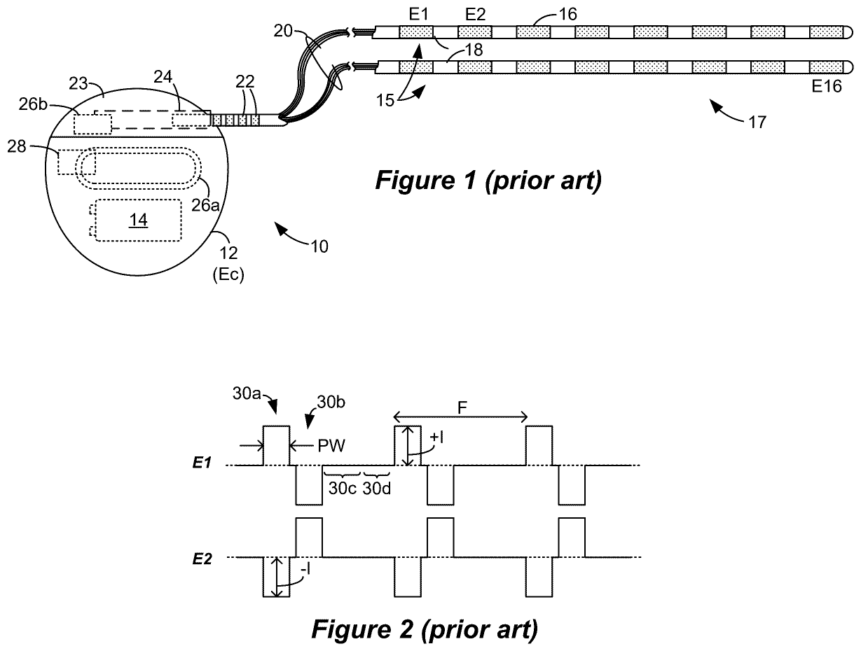

Nonetheless, at least for some patients, SCS therapy would ideally provide complete pain relief without paresthesia—what is often referred to as “sub-perception” or sub-threshold therapy that a patient cannot feel. Effective sub-perception therapy may provide pain relief without paresthesia by issuing stimulation pulses at higher frequencies. Unfortunately, such higher-frequency stimulation may require more power, which tends to drain the battery 14 (FIG. 1) of the IPG 10. Sec, e.g., U.S. Patent Application Publication 2016/0367822. High-frequency stimulation may occur at frequencies of 1500 Hz or above.

The battery 14 used in an IPG 10 may either be rechargeable or non-rechargeable (primary), and typically the power of the stimulation a given patient requires will play a factor in determining which of these types of IPGs will be selected for implantation with that patient. The stimulation a patient requires, and the power that that stimulation will draw in the eventually-implanted IPG, may be generally be known in advance of implantation of an IPG in a patent, and may be determined during an external trial period when an External Trial Stimulator (ETS) is used to provide trial stimulation, as briefly explained earlier.

The use of higher-frequency stimulation, or higher-powered stimulation more generally, can preclude the use of primary cell IPGs for some patients as a practical matter, because a primary cell IPG would need to be replaced too frequently for these patients, which would be inconvenient and subject such patients to additional surgical risks. Such patients would instead be provided with a rechargeable battery implant, which would not need to be surgically replaced as frequently. However, use of a rechargeable-battery IPG is also problematic when a patient requires higher-frequency or higher-power stimulation, because that patient will need to charge the IPG more frequently and/or for longer periods of time, which may be inconvenient. Sec, e.g., U.S. Pat. No. 11,129,996 (describing the use of an external charger to charge a rechargeable-battery IPG). Furthermore, some patients may be unwilling or unable (due to age, disability, etc.) to reliably charge a rechargeable-battery IPG.

Lower-frequency SCS stimulation, or lower-powered stimulation more generally, is therefore desirable. Sub-perception SCS therapies at lower frequencies, and hence lower powers, are known. Sec, e.g., U.S. Pat. No. 10,576,282 and U.S. Patent Application Publication 2021/0299448, which are both incorporated by reference in their entireties. These publications describe the use of sub-perception SCS therapies at frequencies as low as 10 Hz, or even 2 Hz, although the techniques described in these references are somewhat complicated.

Described herein are low-frequency sub-perception algorithms for determining optimal low-frequency and sub-perception spinal cord stimulation for a patient. These algorithms as described provide a number of advantages. First, a spread bipole is preferably used to both to position the stimulation to target a patient's pain, and to provide the resulting therapy. As discussed above, a spread bipole is able to recruit a large area or volume of tissue, and so it easier and less time consuming to target. Further, a spread bipole maintain therapy for longer because it is less prone to becoming untargeted if the electrode array 17 migrates.

Second, the stimulation is provided at very low frequencies, such as 10 Hz or less, and preferably 2 Hz. Further, sub-perception is provided by the use of lower amplitudes and at longer pulse widths than are conventional in typical SCS stimulation therapies. Despite the use of longer pulse widths, the low frequencies and amplitudes used result in determined stimulation therapies for patients that are very low power, and much lower when compared to high frequency stimulation therapies as described above. This is beneficial, and particularly so because it facilitates the use of primary cell IPGs, which as described above can be beneficial for some patients when compared with rechargeable cell IPGs.

Third, therapy is provided by the use of biphasic pulses, and preferably symmetric biphasic pulses. The use of such pulses are preferred for a number of reasons. They naturally provide active charge recovery (although passive charge recovery may also be used if necessary). Further, use of biphasic pulses are beneficial because, as is known, the cathode pole is largely involved in neural tissue recruitment. When a biphasic pulse is used, the positions of the anode and cathode will flip during the pulse's two phases. This effectively doubles the neural tissue that is recruited for stimulation, and thus increases the possibility that a site of pain in the patient's tissue will be covered by a bipole at the correct location. In effect, the symmetric biphasic pulse can be viewed as providing two points of stimulation to the tissue, increasing both coverage and therapy. See U.S. Patent Application Publication 2021/0299448.

While these aspects of the disclosed algorithms are beneficial, they are not all necessarily required. For example, spread bipoles may not necessarily be used; the frequency used can vary; and pulses that are not biphasic or symmetrically biphasic could be used in different useful implementations. Further, the disclosed algorithms can take different forms. Not all steps in the examples of algorithm that follow need to necessarily be performed, and other steps could be added to other implementations. Steps of the disclosed algorithms could also appear in different orders compared to what is illustrated, and the illustrated orders are not intended to be limiting.

The disclosed algorithms are likely used by a clinician to set therapy for the patient, which would most likely involve use of the clinician's programmer, although any external system could be used to perform the algorithm. Once therapy has been determined, the clinician programmer can program the patient's IPG to execute the therapy. Thereafter, the patient may use his external controller to adjust the determined therapy in a limited fashion so as to maintain sub-perception, as discussed further below. The disclosed algorithms can comprise a part of the external system software 96 which renders the GUI 99, and as such may be embodied in a computer readable media, as explained previously.

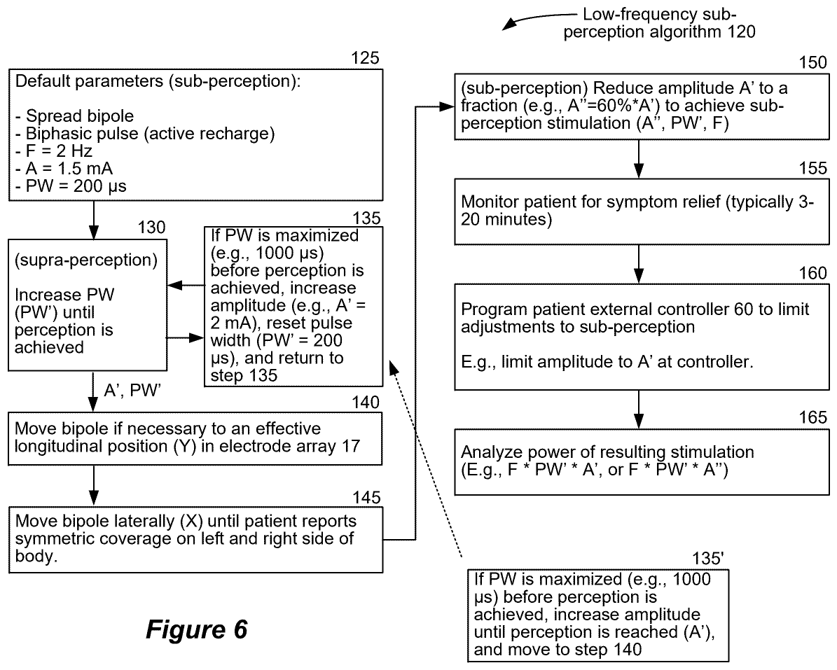

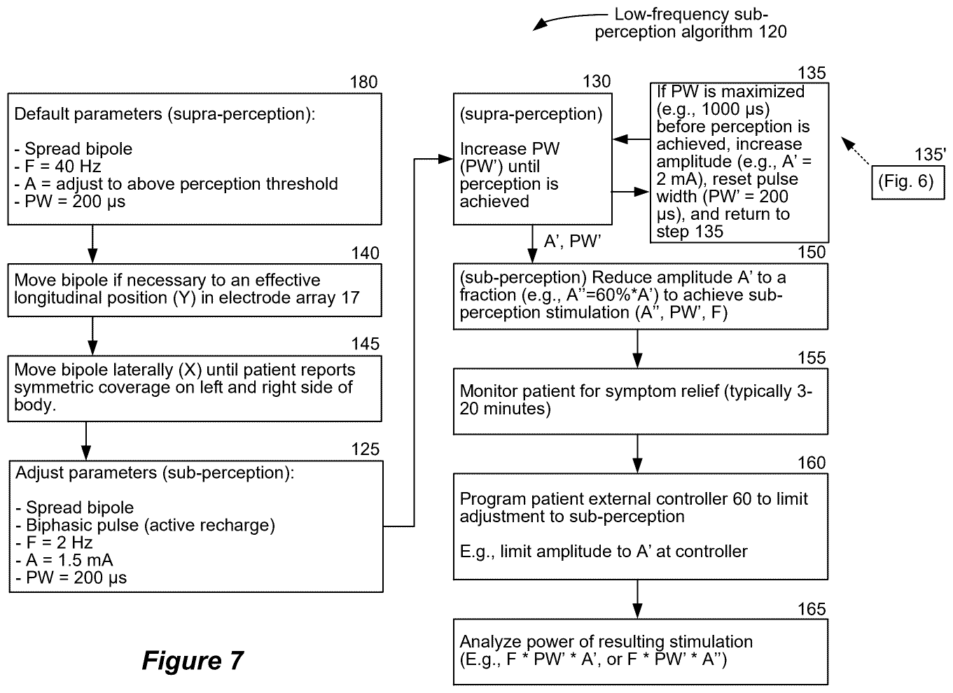

Different examples of the low-frequency sub-perception algorithm 120 are shown in FIGS. 6 and 7. As shown in FIG. 5A, the algorithm 120 can be started by selecting an option 122 on the GUI 99. Selecting this option 122 can automate use of the steps in the algorithm 120 that are discussed subsequently, or can prompt the user to follow these steps in the GUI 99. In this regard, although not shown, option 122 can act as a software “wizard” to automate and sequentially step the user through the different steps of the algorithm 120. While an option like 122 can be useful in practicing the algorithm 120, it is not strictly necessary. The clinician can also perform the algorithm 120 by manually performing the individual steps using the GUI 99.

Use of option 122, and use of the algorithm 120 more generally, can occur during an external trial phase in which the electrode array 17 has been implanted in the patient, but the IPG 10 has not. Use of the algorithm 120 during this phase can be helpful in selecting an IPG 10 (either a primary or rechargeable cell) for later implantation in the patient, as discussed further below. Algorithm 120 however can also be used to determine or adjust stimulation for a patient that has already been implanted with an IPG 10. This is significant, because previously-established stimulation therapies for the patient may not be optimal. For example, such therapies may be too high powered, or not sub-perception (as a patient might prefer), or no longer properly targeting a source of the patient's pain.

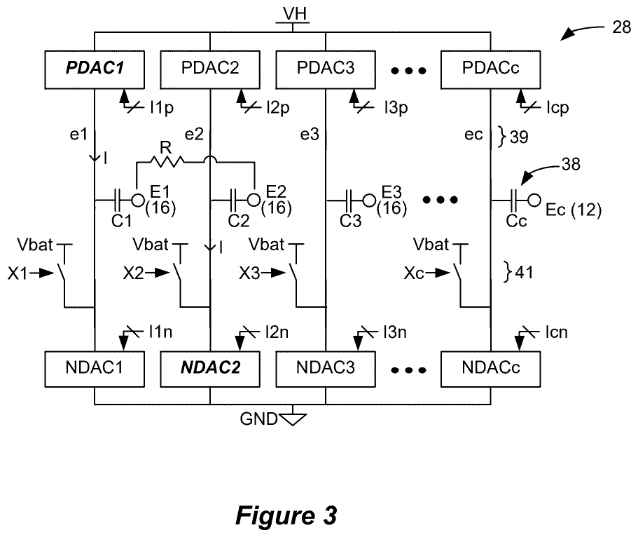

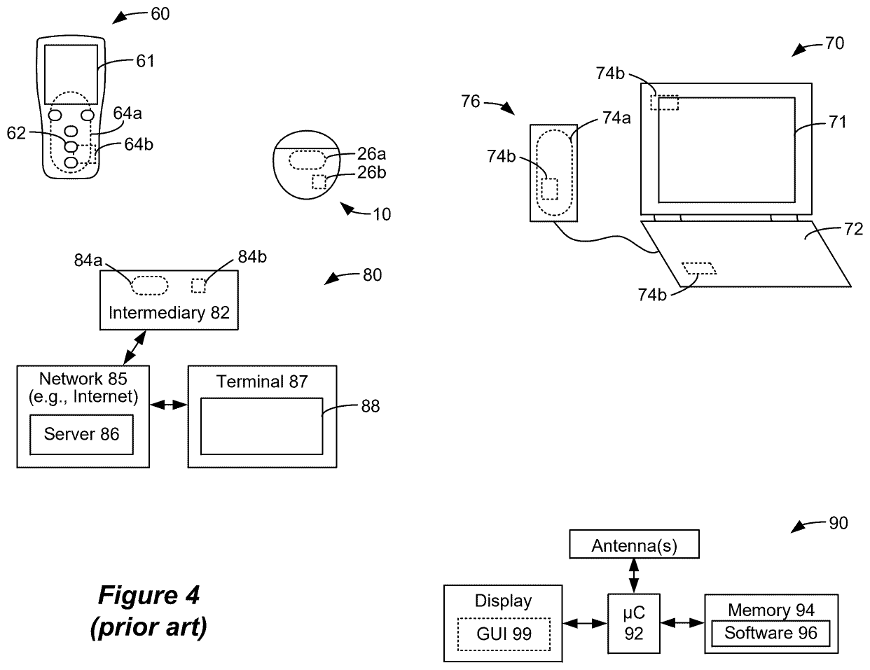

A first example of the algorithm 120 is shown in FIG. 6. At step 125, default stimulation parameters are used to form a spread bipole in the electrode array 17, as described earlier. This spread bipole can have a default focus L (e.g., 16 mm), which may also be adjustable at this stage. At the active electrodes in this spread bipole, biphasic pulses are used which provide active recharge (30b), and most preferably these pulses are symmetric having phases 30a and 30b with equal amplitudes and pulse widths (see FIG. 2). Benefits to the use of spread bipoles and biphasic pulses were described previously.

Other default stimulation parameters are applied to form the bipole at step 125, which preferably results in stimulation that is sub-perception at step 125. For example, a low frequency of 10 Hz or less, and preferably 2 Hz, is used. A generally low amplitude (compared to amplitudes typically used in SCS therapies) is also selected, such as 1.5 mA. A somewhat small pulse width is also used, such as 200 μs. The defaults values are just examples, and other values could be chosen as well. The effect of these default stimulation parameters—and in particular the relatively low amplitude and pulse width—would typically result stimulation that is sub-perception. If the spread bipole is not sub-perceptive at step 125, the amplitude and or pulse width could be further decreased from these default values (e.g., at interface 100, FIG. 5A).

Next, at step 130, the pulse width is incrementally increased (e.g., at interface 100, FIG. 5A) to a point where perception is achieved (PW′), i.e., to a point where the patient is able to feel the stimulation (supra-perception stimulation). This step 130 will typically rely on subjective feedback from the patient, with the patient reporting to the clinician whether the stimulation can be felt each time after the pulse width is increased. During step 130, the user may eventually maximize the pulse width to an upper limit (e.g., PWmax=1000 μs) that the IPG can produce, or that the GUI 99 will allow. Should this occur, an optional step 135 allows the amplitude to be slightly increased (e.g., from A=1.5 to A′=2.0 mA), with the pulse width reset to its initial value (e.g., 200 μs). At this point, the algorithm 120 can return to step 130, where the pulse width is incrementally increased again to attempt to achieve perception. If the pulse width becomes maximized again, the amplitude can be increased again at step 135 if necessary.

If the pulse width is maximized at step 130, the algorithm 120 may optionally employ a different step 135′ in lieu of step 135. In step 135′, the algorithm 120 may keep (and not reset) this maximized pulse width, and increase the amplitude at that maximum pulse width until perception is reached, at which point the algorithm can move to next step 140. Note that steps 135 and 135′ may arrive at different amplitudes A′ and pulse widths PW′: whereas step 135′ will arrive at a pulse width of PW′=PWmax and an amplitude A′=A1, step 135 may arrive at a pulse width PW′=PW2 that is less than PWmax, and an amplitude A′=A2 which is greater than A1.

Notice after step 130 that the determined amplitude (A′) and pulse width (PW′) may vary from the initial default parameters. The determined amplitude A′ may equal the initial default amplitude (e.g., 1.5 mA), or this amplitude may have been increased at steps 135 or 135′. The determined pulse width PW′ will most likely be increased from its default value (e.g., 200 μs), and (depending whether optional steps 130 or 135/135′ are invoked) may comprise a maximum value for the pulse width (PWmax) or some value between the default value and this maximum value. Amplitude (A′) and pulse width (PW′) establish supra-perception stimulation at this point.

Next, at an optional step 140, the bipole is moved to an effective longitudinal position (in the Y direction) in the electrode array 17, using means described earlier (e.g., arrows 106, FIG. 5A). This step 140 is useful to ensure that the bipole is recruiting the site of pain in the patient's tissue. Step 140 may not be necessary in all implementations, or can occur after step 125 and before step 130.

Next, at an optional step 145, the bipole can be moved laterally (in the X direction) in the electrode array 17 until the patient reports perception of the stimulation equally on the left and right sides of his body, i.e., until the stimulation is felt symmetrically by the patient. This step 145 can be useful to ensure that the spread bipole will equally recruit and treat tissue to the left and right of the patient's physiological midline, thus increasing the likelihood that the spread bipole will recruit a site of pain in the patient's neural tissue. In this regard, note that a patient's physiological midline may not correspond to the physical lateral center of the patient. Therefore, even if the leads 15 in the electrode array 17 are implanted in the spinal column perfectly around the physical center of the patient, and even if the bipole is perfectly centered in the electrode array 17, neural tissue to the left and right the patient's physiologic midline may not be recruited to the same degree. Moving the bipole laterally during step 145 to ensure lateral symmetry of perceived neural recruitment corrects for any such non-idealities. Further, centering the stimulation around the physiological midline increases the likelihood that the spread bipole will continue to be effective to treat the patient even if the leads 15 migrate somewhat. Sec, e.g., U.S. Pat. Nos. 10,149,979 and 10,376,702 (discussing spinal cord stimulation in relation to a patient's physiological midline).

Preferably, moving the bipole longitudinally (step 140) and latteraly (step 145) as described are performed when stimulation in the electrode array 17 is supra-perception stimulation that the patient can feel (as occurred earlier at step 130). This makes positioning of the stimulation easier, because the patient can feel the stimulation and provide feedback to the clinician. Having said this, it is not strictly necessary that the stimulation at these steps is supra-perception, and instead the stimulation can be sub-perception as well.

At this point, the stimulation is still supra-perception, but is changed to sub-perception at next step 150. Specifically, at step 150, the amplitude is decreased to a fraction (e.g., 60%) of its current value A′ to achieve stimulation that is sub-perception, arriving at new amplitude A″ (e.g., A″=0.6*A′). Amplitude A′ can be set to A″ using a different pre-determined amount, such as different fraction greater than 0% and less than 100%. Amplitude A′ could also be set to A″ by subtracting a pre-determined amount (Δ) from A′ (e.g., A″=A′−Δ).

At this point, the sub-perception stimulation therapy is optimized for the patient, and applied to the patient. In the example shown (and assuming the amplitude did not need adjustment at steps 135 or 135′, such that A′=A=1.5 mA), the resulting stimulation parameters comprise a frequency F=2 Hz, an amplitude of A″=0.6*1.5=0.9 mA, and a pulse width PW′ of whatever value was determined earlier at step 130. The stimulation is provided by the spread bipole as determined and positioned (steps 140, 145) earlier, and at the default frequency (e.g., F=2 Hz). This stimulation therapy may also at this point be officially stored as a stimulation program in the GUI 99, and transmitted to the patient's external controller (for the patient's use), to the patient's IPG 10, or both. (The program may be limited before it is transmitted, per step 160 discussed further below). If the patient is in an external trial phase, this program may be saved, and later programmed into the IPG 10 once it is implanted into the patient.

Empirical evidence suggests that SCS patients whose stimulation is established using algorithm 120 achieve significant pain relief quickly (as discussed further below), and using stimulation parameters that result in very low power draws. For example, in a small population of patients receiving sub-perception stimulation per algorithm 120 and at a frequency of 2 Hz, an average amplitude A″ of about 1.25 mA, and an average pulse width PW′ of 900 μs, were determined for such patients. Multiplying these numbers provides a relative indication of the power that the stimulation will draw in the IPG, which in this instance (ignoring units) results in a power metric of F*PW′*A″=2*900*1.25=4500. This is a lower power metric than typically results when traditional SCS treatments are used, and a much lower power metric than typically results when high-frequency SCS treatments discussed above are used. The low power metric associated with the sub-perception therapy determined by algorithm 120 results in particular because the frequency (F) and amplitude (A″) are much lower than normal (even though the pulse width may be higher than normal).

Next steps in algorithm 120 are optional but potentially useful. Empirical evidence suggests that patients whose sub-perception stimulation therapies are set per algorithm 120 will exhibit symptom relief (e.g., a reduction in pain) quickly, within 3-20 minutes, as the effects of the sub-perception stimulation washes in. The patient may therefore at this point be monitored over this period of time at step 155 to verify that good symptom relief is achieved.

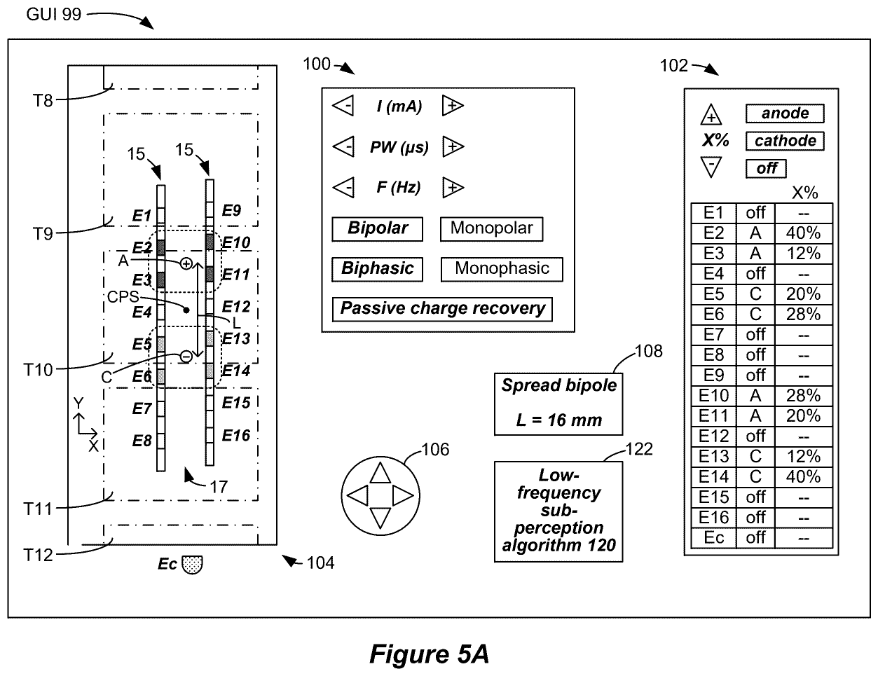

If a good symptom relief is achieved within this time period, the patient's external controller 60 (FIG. 4) may be programmed by the clinician to limit patient adjustments to the determined therapy program within a range that provides the desired sub-perception stimulation (step 160). Because a patient's external controller 60 may be more simple, and only able to adjust the stimulation parameter of amplitude (but not frequency or pulse width), the maximum amplitude the controller 60 can produce may be limited, with the patient able to only adjust the amplitude between 0 and that maximum amplitude. Preferably, this maximum amplitude is set to the amplitude value A′ used (or arrived at) in steps 130/135/135′. In other words, the controller 60 may be programmed with a maximum amplitude equal to the default value used at step 125 (e.g., 1.5 mA, assuming the amplitude was not increased at steps 135 or 135′), or to the increased amplitude used at steps 135 or 135′. This is a logical maximum amplitude to use, because A′ was the highest amplitude that provided sub-perception stimulation at the pulse width (PW′) determined for the stimulation at step 130 (or the lowest amplitude that provided supra-perception stimulation).

Programming the patient's external controller at step 160 can occur in different ways. For example, the maximum amplitude producible by the controller 60 can be programmed by the clinician programmer 70, or the clinician can access special modes of the controller 60 which are not normally visible to the patient (and which may be “hidden” behind entry of a clinician password for example). The therapy program may also be programmed with the maximum amplitude at the clinician programmer 70 before being transmitted to the external controller 60.

Optional step 165 analyzes the resulting power of the sub-perception stimulation arrived at by use of the algorithm 120. This may involve computation of a power metric indicative of the power that the stimulation will draw in the IPG 10. This metric may involve the stimulation parameters the patient is expected to use for therapy (e.g., F*PW′*A″), but could also comprises a worst-case scenario assuming that the patient may increase the amplitude to its programmed maximum A′ as set in step 160 (e.g., F*PW′*A′). If algorithm 120 is run during an external trial phase, power analysis at step 165 may be particularly useful in selecting a type of IPG 10 to be implanted in the patient going forward, and in particular may be useful to determine whether a primary cell IPG can be used for the patient. As described earlier, it can be desirable to select use of a primary cell IPG for at least some patients, but only if the power draw of the stimulation is suitably low. Empirical evidence suggests that the stimulation therapy determined for patients using algorithm 120 is suitably low in power, and would typically allow a primary cell IPG 10 to operate for a period of almost 7 years on average before needing explanation/replacement. This is substantially better than typically reported lifetimes of primary cell IPGs, which average about 3.5 years before explanation is required. The low power stimulation determined by algorithm 120 is also beneficial when executed in a rechargeable IPG, and greatly reduces the charging burden on the patient. Whereas a typical rechargeable cell IPG may be charged about twice weekly, charging is forecasted to only be required about once a month when the low powered stimulation from algorithm 120 is used for stimulation.

FIG. 7 shows another example of low-frequency sub-perception algorithm 120. This algorithm 120 includes some similar steps to those shown and discussed earlier with respect to FIG. 6, and such similar steps are numbered similarly.

First, step 180 again provides default stimulation parameters, but some of these parameters have changed (compare FIG. 6, 125). As before, a spread bipole is used, and a default pulse width of 200 μs is used. However, the frequency at this point is not low (e.g., 2 Hz), but is instead a frequency (e.g., 40 Hz) that is more typically used in traditional SCS therapies. Moreover, it is not critical that a (symmetric) biphasic pulse is used at this point, although that would be logical and typical. Lastly, the amplitude is set to a value above a perception threshold. That is, the amplitude is adjusted upwards to a point that the patient can (comfortably) feel the stimulation. This makes it easier to longitudinally (step 140) and laterally (145) position the stimulation in the electrode array 17. These steps 140 and 145 are otherwise as described earlier with respect to FIG. 6, and may be optional.

Once the stimulation has been positioned in the electrode array 17 (140, 145), the stimulation is adjusted to the default parameters described earlier (FIG. 6, 125). Thus, the amplitude is set to a low value of A=1.5 mA, and the frequency is set to a low value of 2 Hz. Further, the pulses are changed (if necessary) to be biphasic with active recharge, which as before is preferably symmetric. The spread bipole and pulse width can remain the same. As discussed earlier, use of these parameters should establish sub-perception stimulation, and if not certain parameters (e.g., amplitude) could be lowered at this step.

Next, step 130 as described earlier is performed, during which the pulses width is increased (PW′) to a value where supra-perception is achieved. Steps 135 and 135′ as described earlier can also be iteratively performed to increase the amplitude (A′) if the pulse width is maximized before perception is achieved. In short, and as before, an amplitude (A′) and pulse width (PW′) are determined which may vary from the initial default parameters (at step 180).

Next, step 150 as described earlier is performed, during which the amplitude A′ is reduced to a fraction (A″) to achieve sub-perception stimulation. As before, this determines the sub-perception therapy for the patient (e.g., using the spread bipole at F, PW′ and A″). Optional steps 155 (patient monitoring during a wash-in period), 160 (programming the patient's external controller 60 to limit patient adjustments to sub-perception), and 165 (analysis of the resulting power of the stimulation) can occur as described before.

As discussed above, the disclosed algorithms can be performed even before a patient is implanted with an IPG, i.e., during an external trial phase when only the electrode array 17 (leads 15) have been implanted in the patient, with the leads being connected percutaneously to an external trial stimulator (ETS). Such ETS hardware should be construed as a “spinal cord stimulator implanted in a patient” for purposes of this disclosure, even though not fully implanted.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for programming a spinal cord stimulator using an external system in communication with the spinal cord stimulator, the spinal cord stimulator comprising an electrode array comprising a plurality of electrodes implanted in a spinal column of a patient, the method comprising:
(a) accessing a graphical user interface of the external system to cause the spinal cord stimulator to provide stimulation pulses of a first amplitude and a first pulse width from at least one electrode in the electrode array, wherein the stimulation pulses are sub-perception for the patient;
(b) accessing the graphical user interface to cause the spinal cord stimulator to increase the first pulse width to a second pulse width where the stimulation pulses are supra-perception for the patient;
(c) accessing the graphical user interface to cause the spinal cord stimulator to decrease the first amplitude to a second amplitude where the stimulation pulses are sub-perception for the patient; and
(d) providing therapeutic sub-perception stimulation to the patient via the stimulation pulses at the second amplitude and at the second pulse width.

2. The method of claim 1, wherein the stimulation pulses have a first frequency of 10 Hz or less.

3. The method of claim 1, wherein the stimulation pulses form a bipole in the electrode array.

4. The method of claim 3, further comprising, before step (a), accessing the graphical user interface of the external system to cause the spinal cord stimulator to provide stimulation pulses which are supra-perception for the patient.

5. The method of claim 4, further comprising moving the bipole in the electrode array.

6. The method of claim 5, wherein the bipole is moved longitudinally in the electrode array to address a symptom of the patient.

7. The method of claim 5, wherein the bipole is moved laterally in the electrode array to a point where the patient perceives symmetric stimulation on a left and right side of his body.

8. The method of claim 3, wherein the stimulation pulses form a spread bipole configured to approximate a linear electric field in tissue of the patient.

9. The method of claim 1, wherein the stimulation pulses comprise biphasic pulses comprising a first phase of a first polarity and a second phase of a second polarity.

10. The method of claim 9, wherein the first and second phases are symmetric.

11. The method of claim 9, wherein the first and second phases are actively driven by stimulation circuitry in the spinal cord stimulator.

12. The method of claim 1, wherein the steps are performed in order.

13. The method of claim 1, wherein in step (b), if the second pulse width is maximized before the stimulation pulses are supra-perception for the patient, adjusting the first amplitude to a higher value where the stimulation pulses are supra-perception for the patient.

14. The method of claim 1, wherein in step (b), if the second pulse width is maximized before the stimulation pulses are supra-perception for the patient, adjusting the first amplitude to a higher value, setting the pulse width to the first pulse width, and repeating step (b).

15. The method of claim 1, wherein performance of the method is enabled via selection of an option of the graphical user interface.

16. The method of claim 1, wherein in step (c) the first amplitude is decreased to the second amplitude by a pre-determined amount.

17. The method of claim 1, wherein step (d) comprises providing therapeutic sub-perception stimulation to the patient via the stimulation pulses at the second amplitude, the second pulse width, and the first frequency.

18. The method of claim 1, further comprising programming a patient external controller with the therapeutic sub-perception stimulation.

19. The method of claim 18, wherein the patient external controller is configured to allow the patient to adjust the second amplitude of the therapeutic sub-perception stimulation, wherein adjustment of the second amplitude is limited to a maximum comprising the first amplitude.

20. An external system configured for communication with a spinal cord stimulator, the spinal cord stimulator comprising an electrode array comprising a plurality of electrodes implanted in a spinal column of a patient, the external system comprising a graphical user interface and control circuitry configured to:
(a) receive via the graphical user interface a first input to cause the spinal cord stimulator to provide stimulation pulses of a first amplitude and a first pulse width from at least one electrode in the electrode array, wherein the stimulation pulses are sub-perception for the patient;
(b) receive via the graphical user interface a second input to cause the spinal cord stimulator to increase the first pulse width to a second pulse width where the stimulation pulses are supra-perception for the patient; and
(c) receive via the graphical user interface a third input to cause the spinal cord stimulator to decrease the first amplitude to a second amplitude where the stimulation pulses are sub-perception for the patient, whereby the spinal cord stimulator provides therapeutic sub-perception stimulation for the patient via the stimulation pulses at the second amplitude and at the second pulse width.
